(19) United States
(12) Patent Application Publication
(10) Pub. No.: US 2024/0310360 A1
(43) Pub. Date: Sep. 19, 2024

(54) METHOD OF EVALUATING IMMUNE RESPONSE OF CELL GROUP TO TEST SUBSTANCE

(71) Applicant: CANON KABUSHIKI KAISHA, Tokyo (JP)

(72) Inventors: KOUICHI KATO, Kanagawa (JP); TSUTOMU HONMA, Tokyo (JP); TAKANORI YODA, Kyoto (JP)

(21) Appl. No.: 18/605,531

(22) Filed: Mar. 14, 2024

(30) Foreign Application Priority Data
Mar. 16, 2023 (JP) 2023-042238

(51) Int. Cl. G01N 33/50; G01N 21/64; G06T 7/00

(52) U.S. Cl. CPC G01N 33/5047; G01N 21/6486; G06T 7/0014; G06T 2207/10024; G06T 2207/10064; G06T 2207/30024

(57) ABSTRACT

A method of evaluating an immune response of a cell group to a test substance, the method comprising evaluating the immune response of the cell group to the test substance based on a comparison between autofluorescence information of a first sample prepared by adding the test substance to the cell group and autofluorescence information of a second sample, the second sample serving as a control for the first sample.

### BACKGROUND

### Field of the Disclosure

The present disclosure relates to a method of evaluating an immune response of a cell group to a test substance, an information method, an information processing apparatus, an information processing program, and a system for evaluating an immune response of a cell group to a test substance.

### Description of the Related Art

Animals including humans are equipped with a mechanism of defense through inactivation of a foreign substance, such as a pathogen or a toxin, which enters the body, or a cancer cell, that is, an immune system. The immune system includes an early response, which is a quick reaction to a non-specifically recognized foreign substance, that is, innate immunity, and an “antigen-specific immune response” (also called acquired immunity or adaptive immunity), which is a reaction to a foreign substance or a part thereof that is specifically recognized as an “antigen.”

An antigen-specific immune response involves T cells, B cells, and other immune cells, and is equipped with a mechanism of storing information of an antigen encountered in the past and responding quickly upon re-encounter with the same antigen. Accordingly, evaluation of an antigen-specific immune response of an immune cell is useful in examinations and studies of such diseases as infectious diseases, cancers, allergies, and autoimmune diseases, evaluation of a medicinal agent that acts on the immune system as an antigen, such as a vaccine, and the like.

As a typical method of evaluating an antigen-specific immune response of a cell, there has been known a method of stimulating a T cell or another immune cell with an antigen and detecting an immune effector molecule (for example, a cytokine such as INF-γ) secreted as a result of a response of the immune cell to the antigen.

For example, enzyme-linked immunospot (ELISpot) assay is a method in which an antibody bound to a surface of a solid phase that is an antibody against an immune effector molecule is used to capture immune effector molecules secreted from cells cultured on the solid phase and, after the cells are removed and the surface of the solid phase is washed, the molecules are visualized with use of a labeled antibody, to thereby count the cells that have secreted the molecules (J. Immunol. Methods, 128, 65-73, 1990). FluoroSpot assay is a method of evaluating an antigen-specific immune response with the same mechanism as the mechanism of ELISpot, except that a fluorescently labeled antibody is used to visualize the immune effector molecules, and enables simultaneous detection of a plurality of types of immune effector molecules by using a plurality of labeled antibodies different from one another in fluorescence wavelength (Cells, 3(4), 1102-1115, 2014). ELISpot assay and FluoroSpot assay are widely used in medical tests and drug development because of high sensitivity and capability to evaluate on a cell-by-cell basis. For example, a test to see if there is tuberculosis infection and a check of effects of materials that are candidates for a vaccine use ELISpot assay or FluoroSpot assay.

As described above, ELISpot assay and FluoroSpot assay are excellent methods of evaluating an antigen-specific immune response of a cell, but are cumbersome in terms of operation and require proficiency at work. Further, ELISpot assay and FluoroSpot assay require cultivation of cells until immune effector molecules are secreted in an amount sufficient for visualization and, as a result, usually take 1 to 2 days or longer for a testing time. A simpler and quicker evaluation method to replace ELISpot assay and FluoroSpot assay has accordingly been sought in a case of an infectious disease or the like in which testing is required to be quick and a case of drug development in which a large number of candidate substances or specimens are required to be evaluated.

For example, in an existing way of determining an effect of a vaccine, testing by ELISpot assay in which a human peripheral blood mononuclear cell (PBMC) is immingled with an antigen takes as long a time as 24 hours to 48 hours. However, testing that takes a long time equals not only a poor throughput of results but also a possibility of a change of properties of cells due to a long time of cultivation.

### SUMMARY OF THE INVENTION

An object of the present disclosure is to provide a simple and quick method of evaluating an immune response of a cell group to a test substance.

The inventors of the present disclosure have searched for a change of a cell that appears earlier than secretion of immune effector molecules after the cell is stimulated with an antigen and that is detectable with ease. As a result, it has been found out that autofluorescence of the cell, namely, fluorescence emitted from an endogenous fluorescent substance possessed by the cell when the cell is irradiated with excitation light having a specific wavelength, changes in a short time after the stimulation with the antigen. The inventors of the present disclosure have further found out that this change correlates with an antigen-specific immune response of the cell, and thus have made the present disclosure.

That is, according to aspects of the present disclosure, there is provided a method of evaluating an immune response of a cell group to a test substance, the method comprising evaluating the immune response of the cell group to the test substance based on a comparison between: first autofluorescence information which is autofluorescence information of a first sample, the first sample being prepared by adding the test substance to the cell group; and second autofluorescence information which is autofluorescence information of a second sample, the second sample serving as a control for the first sample. Further features of the present disclosure will become apparent from the following description of embodiments with reference to the attached drawings.

### BRIEF DESCRIPTION OF THE DRAWINGS

FIG. 1 is a schematic diagram for illustrating a cell group that reacts specifically to a test substance.

FIG. 2 is a conceptual diagram for illustrating an embodiment of the present disclosure.

FIG. 3A is a graph showing an example of a case in which it is evaluated that there is an immune response in the embodiment of the present disclosure.

FIG. 3B is a graph showing another example of the case in which it is evaluated that there is an immune response in the embodiment of the present disclosure.

FIG. 3C is a graph showing still another example of the case in which it is evaluated that there is an immune response in the embodiment of the present disclosure.

FIG. 3D is a graph showing yet still another example of the case in which it is evaluated that there is an immune response in the embodiment of the present disclosure.

FIG. 4 is a diagram for illustrating a system configuration of an image pickup and analysis system.

FIG. 5 is a diagram for illustrating a functional configuration of a part 1A of the image pickup and analysis system of FIG. 4.

FIG. 6 is a diagram for illustrating a functional configuration of a part 2A of the image pickup and analysis system of FIG. 4.

FIG. 7 is a scheme chart of image analysis in which an image for cell region extraction and an autofluorescence image that are photographed by the image pickup and analysis system are used, and an image processing device takes information out of the images, plots, visualizes, and performs clustering.

FIG. 8 is an example of an image that is a bright-field photograph of PBMCs photographed by the image pickup and analysis system.

FIG. 9 is an example of an image that is a fluorescence photograph of PBMCs photographed by the image pickup and analysis system.

FIG. 10A is an example of measurement of an FAD/NADH ratio calculated from an autofluorescence imaging result 1 hour after addition of a test substance varied in reactivity to PBMCs.

FIG. 10B is another example of measurement of the FAD/NADH ratio calculated from an autofluorescence imaging result 1 hour after addition of the test substance varied in reactivity to PBMCs.

FIG. 10C is still another example of measurement of the FAD/NADH ratio calculated from an autofluorescence imaging result 1 hour after addition of the test substance varied in reactivity to PBMCs.

FIG. 11A is an example of measurement of an FAD/NADH ratio 1 hour after addition of a highly reactive test substance.

FIG. 11B is another example of measurement of the FAD/NADH ratio 4 hours after addition of the highly reactive test substance.

FIG. 12A is an example of measurement of changes with time of an FAD/NADH ratio up through 24 hours since addition of a highly reactive test substance.

FIG. 12B is another example of measurement of changes with time of the FAD/NADH ratio up through 24 hours since addition of the highly reactive test substance.

FIG. 12C is still another example of measurement of changes with time of the FAD/NADH ratio up through 24 hours since addition of the highly reactive test substance.

### DESCRIPTION OF THE EMBODIMENTS

An embodiment according to the present disclosure provides a method of evaluating an immune response of a cell group to a test substance, the method including evaluating the immune response of the cell group to the test substance based on a comparison between: first autofluorescence information which is autofluorescence information of a first sample, the first sample being prepared by adding the test substance to the cell group; and second autofluorescence information which is autofluorescence information of a second sample, the second sample serving as a control for the first sample.

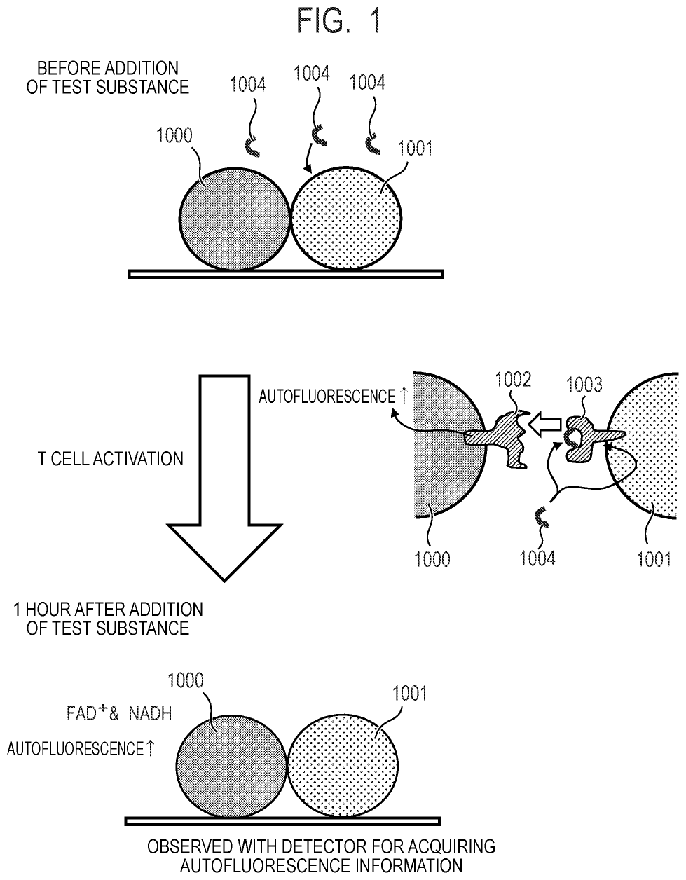

An example of a cell group that has an immune response to a test substance is illustrated in FIG. 1. The cell group of FIG. 1 includes a T cell 1000 and an antigen presenting cell (APC) 1001. A test substance 1004 is presented on a major histocompatibility complex (MHC) 1003 on the antigen presenting cell 1001. When the T cell has a T cell receptor (TCR) 1002 specific to a complex of the test substance 1004 and the major histocompatibility complex 1003, those are bound together to activate the T cell 1000.

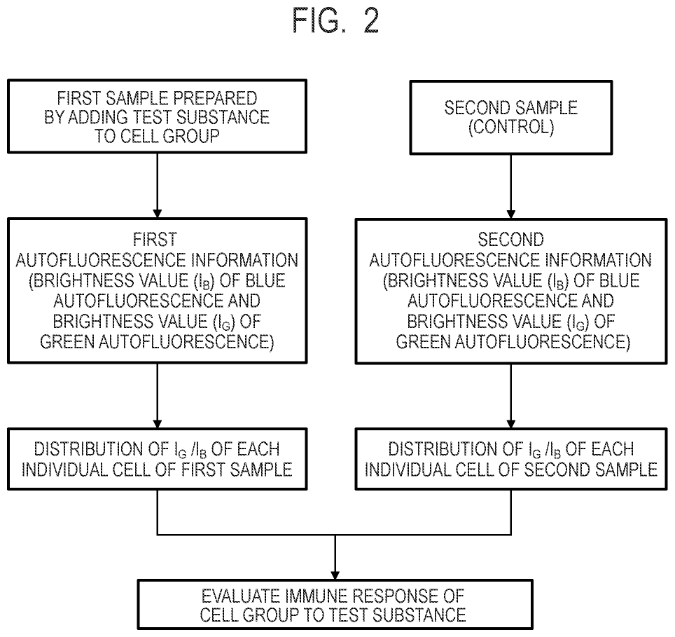

A conceptual diagram of this embodiment is illustrated in FIG. 2.

According to the method of this embodiment, an immune response of a cell group to a test substance is evaluated based on a comparison between the first autofluorescence information, which is autofluorescence information of the first sample prepared by adding the test substance to the cell group, and the second autofluorescence information, which is autofluorescence information of the second sample serving as a control for the first sample.

The autofluorescence information can include information about a brightness value (I_{B}) of blue autofluorescence and a brightness value (I_{G}) of green autofluorescence.

The autofluorescence information also includes information on a value (I_{G}/I_{B}) of a ratio between the brightness value (I_{B}) of blue autofluorescence and the brightness value (I_{G}) of green autofluorescence of each individual cell of each sample, and the first autofluorescence information and the second autofluorescence information can be compared based on distribution of I_{G}/I_{B} of each individual cell of each cell group.

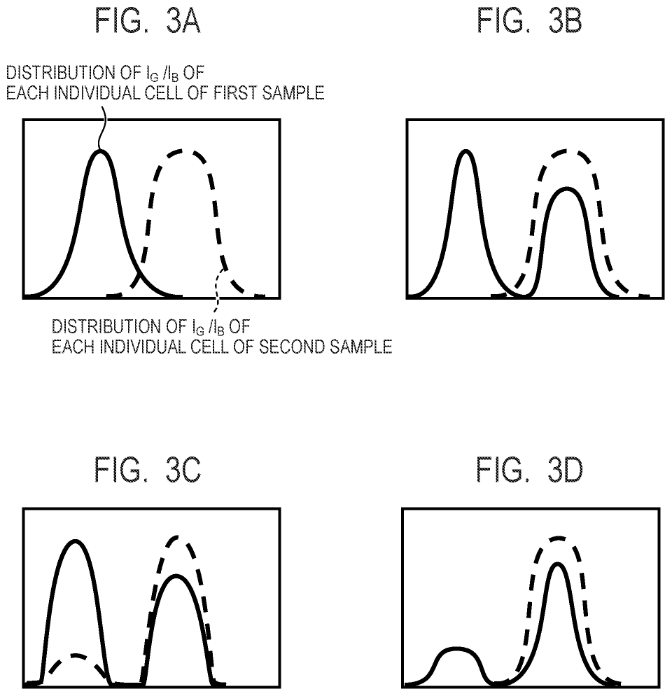

Based on this comparison, as in illustration of FIG. 3A to FIG. 3D, for example, it can be evaluated that the cell group has an immune response to the test substance, or that the immune response of the cell group is more intense compared to the control in the following cases. In FIG. 3A to FIG. 3D, the solid line represents distribution of I_{G}/I_{B} of each individual cell of the first sample, and the dotted line represents distribution of I_{G}/I_{B} of each individual cell of the second sample.

The evaluation described above is given in a case in which an average value, a median value, or a mode value of I_{G}/I_{B} of cells of the first sample is smaller than that of the second sample (FIG. 3A), or a case in which the first sample exhibits multimodality whereas the second sample is unimodal (FIG. 3B and FIG. 3D), or a case in which the first sample and the second sample both exhibit multimodality but differ from each other in the shape of modes (FIG. 3C).

In a review of the difference described above, a length of time elapsed since addition of the test substance and a concentration of the test substance may be taken into consideration. Further, intensity of the immune response may be evaluated based on the difference described above, the elapsed time, and the concentration of the test substance.

The second sample may be prepared by not adding the test substance to the cell group, or adding the test substance to the cell group in an amount different from the amount of the test substance in the first sample, or adding a substance that is a substitute for the test substance to the cell group, or varying the time elapsed since the addition of the test substance to the cell group from that of the first cell group.

The first sample and the second sample are not always required to be prepared separately from each other, and the second sample can be a sample collected before the addition of the test substance in preparation of the first sample, or a sample collected not long after the addition of the test substance in preparation of the first sample of the test substance in preparation of the first sample.

A specific configuration example of this embodiment is given below, but the present disclosure is not limited to a method described below.

### (Cell Group)

There are no particular limitations on a cell group to be provided for the evaluation method of this embodiment, as long as an antigen-specific immune cell can be evaluated. A preferred cell group includes at least one of a T cell, a B cell, or other lymphocytes. A particularly preferred cell group is a peripheral blood mononuclear cell (PBMC). Alternatively, a PBMC heightened in lymphocyte proportion by preculturing a PBMC, removing adhered cells, and collecting floating lymphocytes may also be used. Thus, utilization of a cell obtained by temporary culture of an immune cell taken out of a living body is not limited as well.

Further, any type of cell taken out of a PBMC by an existing method, for example, a specific immune cell such as a CD8-positive cell, a CD-4 positive cell, or a B cell, may be used.

The number of cells or a cell concentration of the cell group to be provided for the evaluation method of this embodiment is set within a range favorable for a sample that is used. For example, in a case of evaluating an antigen-specific immune response of a T-cell in a PBMC, stimulation of the T cell with a test substance requires contact with a cell that presents an antigenic site derived from the test substance, and a cell density at which cells are in contact with one another is accordingly required. Specifically, a cell density of around 1×10⁶ cells/cm² is preferred for a case of applying stimulation in static culture. Alternatively, T cells and other lymphocytes which are culturable in a floating state can be brought into contact with one another at a lower cell density by adjusting a condition for furthering movement of cells, for example, a shaking condition in a case of shaking culture. Still alternatively, use of a culture vessel with a non-flat bottom such as a microplate with a V bottom or a U bottom enables culture with cells being in contact with one another, irrespective of cell density.

For example, a test substance is added to a cell group that is a group of PBMCs or similar cells and, after 1 hour or so, autofluorescence derived from reduced nicotinamide adenine dinucleotide (NADH) and/or reduced nicotinamide adenine dinucleotide phosphate (NADPH) possessed by the cells, and autofluorescence derived from oxidized flavin adenine dinucleotide (FAD⁺) possessed by the cells are measured. Reactivity of the cell group to the test substance can be evaluated from a change in fluorescence ratio (an FAD/NADH ratio) of those autofluorescence components.

### (Test Substance)

Antigens, antigenic proteins, antigenic peptides, vaccines, and pathogens are given as examples of test substances. Peptides derived from those test substances are also given as examples of test substances.

What is capable of stimulating a TCR of a CD8-positive T cell is an MHC class 1 to which a peptide derived from a test substance and formed from 8-10 amino acids is bound. What is usable as a test substance is a peptide that is derived from a test substance and that binds to an MHC class 1 prepared in advance. As another form of test substance, a test substance formed from a protein or a polypeptide is usable, and this test substance may be introduced into a cell to receive processing inside the cell so that a peptide derived from the test substance is bound to an MHC molecule for use in evaluation. As still another form of test substance, a test substance holding antigen genetic information (for example, a messenger RNA vaccine) is usable. This test substance may be introduced into a cell to express a protein and receive processing inside the cell so that the processed test substance is bound to an MHC for use in evaluation.

What is capable of stimulating a TCR of a CD4-positive T cell is an MHC class 2 to which a peptide derived from a test substance and formed from 12-24 amino acids is bound. In evaluation of a CD4-positive T cell as well, a peptide that is derived from a test substance and that binds to an MHC class 2 prepared in advance is usable, and a test substance formed from a protein or a polypeptide and a test substance holding antigen genetic information are usable as described above. However, an MHC class 2 is expressed only on an antigen presenting cell, and coexistence with a cell expressing an MHC class 2 is accordingly required.

### (Autofluorescence and Means for Measuring Autofluorescence)

Autofluorescence of a cell that is measured in this embodiment is fluorescence emitted by an endogenous fluorescent substance produced in the cell, such as NADH and/or NADPH (hereinafter, shortened as “NAD(P)H” in some cases), flavins (FAD⁺ and the like), collagen, fibronectin, tryptophan, and folic acid. However, fluorescence emitted by endogenous fluorescent substances other than those may be measured.

In Examples, glucose metabolic activity is evaluated by measuring autofluorescence of endogenous NAD(P)H and flavins (FAD and the like). For example, when glucose metabolism of a T cell included in a PBMC is activated by an antigen-specific immune response, NAD(P)H produced from a glycolytic system and a citric acid cycle increases and an increase in autofluorescence is consequently observed. A glucose metabolic state changes with time and, with an electron transport system in action, a combination of a decrease in NAD(P)H and an increase in FAD⁺ sometimes happens. A change in glucose metabolic state indicating an activation state of a cell can thus be measured as a difference or a change in autofluorescence caused by a change in amount of FAD⁺ and NAD(P)H in the cell, or as a difference or a change in ratio (the FAD/NADH ratio) of those autofluorescence components.

Any means including existing means such as hitherto known fluorescence microscopes and flow cytometers are useable for measurement of autofluorescence, as long as the autofluorescence of a cell described above can be measured. The above-mentioned means capable of measuring autofluorescence of a large number of cells simultaneously and less invasively is preferred. Means capable of simultaneously measuring fluorescence components of a plurality of wavelengths is further preferred because FAD⁺ and NAD(P)H, for example, differ from each other in the wavelength of emitted fluorescence. As such autofluorescence measuring means, for example, a fluorescence measurement device and a fluorescence image acquisition method that are described later are usable in a favorable manner.

To measure autofluorescence, a cell is irradiated with light having a wavelength capable of exciting an endogenous fluorescent substance that is a subject, and fluorescence emitted as a result is measured. For example, when the subject endogenous fluorescent substance is NAD(P)H, a cell is irradiated with excitation light having a center wavelength in a range of from 320 nm to 370 nm, and blue fluorescence emitted as a result and having a wavelength of 400 nm or more, preferably, blue fluorescence in a range of from 420 nm to 500 nm, is measured. When the subject endogenous fluorescent substance is FAD⁺, a cell is irradiated with excitation light having a center wavelength in a range of from 360 nm to 490 nm, and green fluorescence emitted as a result and having a wavelength of 500 nm or more, preferably, green fluorescence in a range of from 500 nm to 580 nm, more preferably, green fluorescence in a range of from 520 nm to 580 nm, is measured.

For example, a test substance is added to a group of immune cells such as PBMCs to be evaluated, and after 1 hour or so, autofluorescence derived from reduced nicotinamide adenine dinucleotide (NADH) and/or reduced nicotinamide adenine dinucleotide phosphate (NADPH) possessed by the cells, and autofluorescence derived from oxidized flavin adenine dinucleotide (FAD⁺) possessed by the cells are measured. Reactivity of the immune cell group to the test substance can be evaluated from a change in fluorescence ratio (the FAD/NADH ratio) of those autofluorescence components.

### (Analysis and Visualization)

As a method of analysis and visualization of acquired autofluorescence information of cells, for example, a method in which types of information on each individual cell such as information on brightness of autofluorescence at respective wavelengths and cell size information are each plotted along one axis to be visualized as a one-dimensional, two-dimensional, or three-dimensional graph, or a multidimensional graph having more than three dimensions is usable.

To describe in more detail, a method of visualizing with use of a histogram in the case of one dimension, and with use of a scatter diagram in the case of two dimensions and three dimensions is useable. Instead of a histogram and a scatter diagram, a probability density function plot for kernel density estimation or the like, a heat map, and the like may be used to visualize information on each individual cell.

Cell shape information is information of a parameter capable of depicting a shape of a cell. Information about a cell size, a cell shape, and a thickness of the cell are given as an example, but parameters other than those are also usable.

### (Evaluation or Determination Method)

Examples of a method of determining an immune response of a cell group to a test substance include the following.

For example, a cell sample is divided into two to be processed under two conditions: one with addition of the test substance (a first sample) and the other without the addition (a control: a second sample), and autofluorescence of the first sample and autofluorescence of the second sample are measured after a certain length of time. Ratios (FAD/NADH ratios) of autofluorescence derived from FAD to autofluorescence derived from NADH of the two conditions are compared and, from presence or absence of, or a magnitude of, a difference between the samples, presence or absence of, or intensity of, an antigen-specific immune response is evaluated or determined. To give another example, for cells processed in the same manner as the manner described above, the presence or absence of, or the degree of intensity of, the antigen-specific immune response may be evaluated or determined based on a magnitude of a difference in brightness of the autofluorescence that is caused by FAD⁺ and/or NAD(P)H, instead of the FAD/NADH ratios. A specific example of the determination is described in Examples.

There are no limitations on the certain length of time as long as evaluation of immune cells are executable.

In a case of a test substance that binds directly to an MHC, stimulation can be applied quickly to the immune cells and, accordingly, the certain length of time in this case is, for example, from 10 minutes to 24 hours, preferably, from 30 minutes to 4 hours. It is a known fact that an intercellular signal is transmitted within a few seconds to a few minutes to a T cell stimulated with an antigen. Accordingly, a change is thought to start after a few minutes from the processing of the cells with the test substance in the present evaluation method as well.

In a case in which a test substance is introduced into an antigen presenting cell once and, after receiving processing inside the cell, presented to a cell surface, a processing time optimum for the introduced test substance is to be set newly each time.

As a further embodiment of the present disclosure, there is provided an image pickup and analysis system for evaluating an immune response of a cell group to a test substance, the image pickup and analysis system including: an image acquisition device including an irradiation unit configured to irradiate with excitation light of from 320 nm to 490 nm; and an image processing device including a display unit, the image acquisition device including means for acquiring an autofluorescence image of a first sample prepared by adding the test substance to the cell group and an autofluorescence image of a second sample which is a control for the first sample, the image processing device including means for generating, from the autofluorescence images, first autofluorescence information which is autofluorescence information of the first sample and second autofluorescence information which is autofluorescence information of the second sample, the display unit displaying a result of comparing the first autofluorescence information and the second autofluorescence information.

A specific configuration example of this embodiment is described but the present disclosure is not limited to a method described below.

(Configuration of Image Pickup and Analysis System which is Detector for Acquiring Autofluorescence Information)

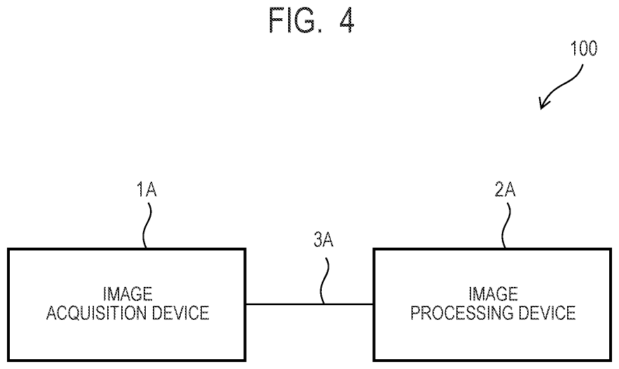

An example of an overall apparatus configuration of an image pickup and analysis system 100 according to this embodiment is illustrated in FIG. 4. The image pickup and analysis system acquires images of cells, calculates a change of each of autofluorescence components having different wavelengths by analyzing the acquired images, and displays the autofluorescence information.

As illustrated in FIG. 4, the image pickup and analysis system 100 is formed by connecting an image acquisition device 1A and an image processing device 2A to each other through an interface such as a cable 3A so as to enable data transmission and reception therebetween. A connection method between the image acquisition device 1A and the image processing device 2A is not particularly limited. For example, the image acquisition device and the image processing device may be connected to each other by a local area network (LAN), or may be connected to each other in a wireless manner.

The image acquisition device 1A acquires an image of cells on a cell culture vessel that is placed on a placement stage, and transmits the image to the image processing device 2A.

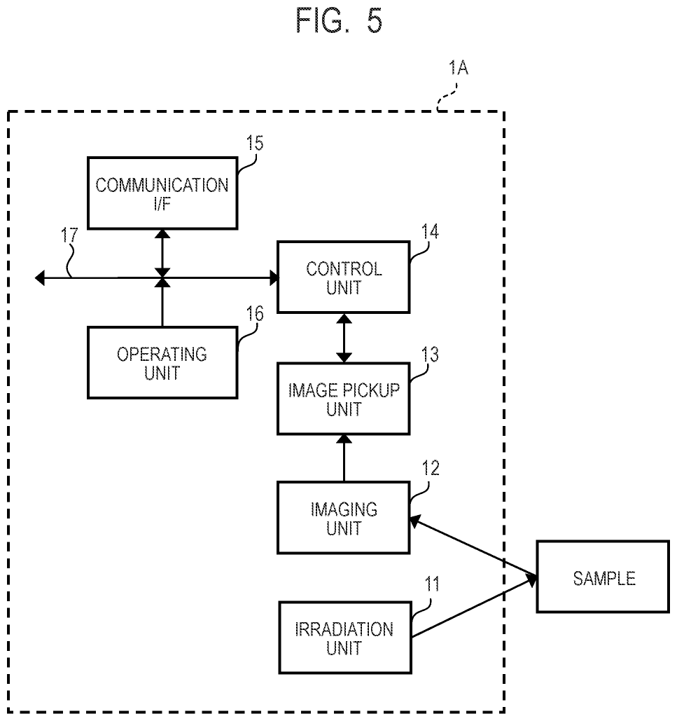

As illustrated in FIG. 5, the image acquisition device 1A includes an irradiation unit 11, an imaging unit 12, an image pickup unit 13, a control unit 14, a communication I/F 15, an operating unit 16, and the like, and the respective units are connected through a bus 17. The irradiation unit 11 is formed of a light source filter or the like for fluorescence observation and a white light source for cell morphology observation, and irradiates a cell group placed on the placement stage with light. The imaging unit 12 is formed of a lens, a filter, and the like, and forms an image of transmitted light, reflected light, or fluorescence that is emitted from the cells in accordance with the applied light. The image pickup unit 13 is a camera that includes a complementary MOS (CMOS) sensor and the like, and picks up an image to be formed on an imaging surface by the imaging unit to generate digital image data (image data of R, G, and B) on the image. The control unit 14 includes a central processing unit (CPU), a random access memory (RAM), and the like, and executes various kinds of processing in cooperation with various programs installed therein. The communication I/F 15 transmits the generated image data on the image to the image processing device 2A. In this embodiment, the image acquisition device 1A includes a bright-field unit in which irradiation means and imaging means that are suitable for bright-field observation are combined, and a fluorescence unit in which irradiation means and imaging means that are suitable for fluorescence observation are combined. The bright field or the fluorescence can be switchable by switching the unit.

The image processing device 2A is an image processing device that analyzes an image transmitted from the image acquisition device 1A, to thereby calculate a feature amount quantitatively indicating an expression level of a specific biological substance in an observation subject cell, and outputs the calculated feature amount.

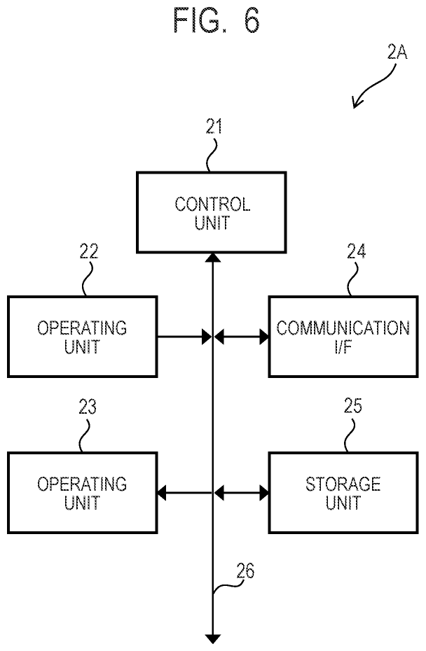

FIG. 6 is an illustration of a functional configuration of the image processing device. The image processing device 2A includes a control unit 21, an operating unit 22, a display unit 23, a communication I/F 24, a storage unit 25, and the like, and the respective units are connected through a bus 26.

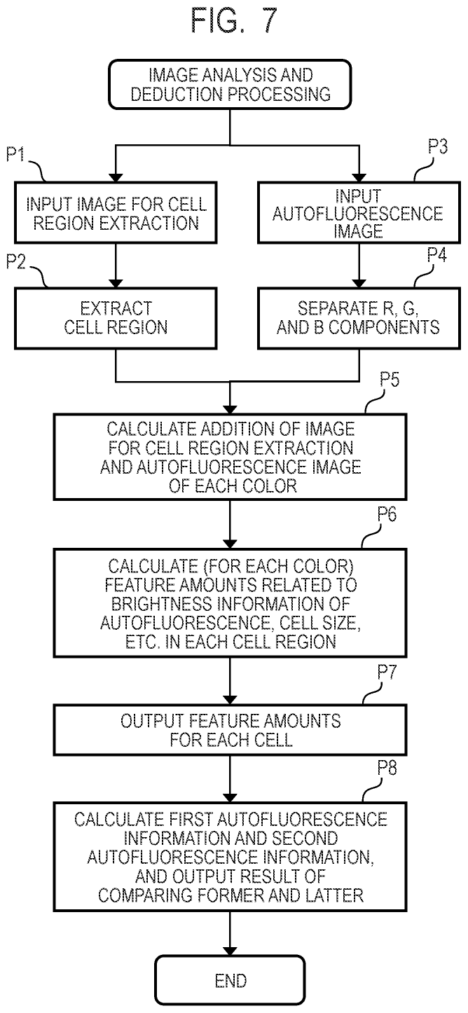

The control unit 21 includes a central processing unit (CPU), a random access memory (RAM), and the like. Various kinds of processing are executed in cooperation with various programs stored in the storage unit 25, and operations of the image processing device are centrally controlled. For example, the control unit 21 executes image analysis processing (see FIG. 7) in cooperation with a program stored in the storage unit 25, and thus implements functions as extraction means, fluorescent bright spot extraction means, region estimation means, feature amount calculation means, and determination means.

The operating unit 22 includes a keyboard provided with character input keys, number input keys, various function keys, and the like and a pointing device such as a mouse, and outputs a pressing signal of a key pressed on the keyboard and an operation signal generated by the mouse to the control unit 21 as input signals.

The display unit 23 includes, for example, a monitor such as a cathode ray tube (CRT) or a liquid crystal display (LCD), and displays various screens in accordance with an instruction of a display signal input from the control unit 21. In this embodiment, the display unit 23 functions as output means which outputs the calculated feature amount and further outputs a comparison result.

The communication I/F 24 is an interface for transmitting and receiving data to and from an external device such as the image acquisition device 1A. The communication I/F 24 functions as input means for a bright-field image and a fluorescence image. In this embodiment, the communication I/F 24 functions as input means.

The storage unit 25 is formed of, for example, a hard disk drive (HDD) or a semiconductor-based nonvolatile memory. The storage unit 25 stores various programs, various kinds of data, and the like as described above. For example, the storage unit 25 stores various kinds of data such as a magnification table to be used in image analysis processing.

In addition, the image processing device 2A may include a LAN adapter, a router, and the like and may be configured to be connected to an external device through a communication network such as a LAN.

The image processing device 2A in this embodiment performs analysis through use of the image for cell region extraction and the autofluorescence image that have been transmitted from the image acquisition device 1A.

The autofluorescence image is an image obtained by irradiating the cells with excitation light having a predetermined wavelength in the image acquisition device 1A to cause a cell-endogenous fluorescent substance to emit light, and subjecting the emitted light which is fluorescence to magnification, imaging, and photographing through a cut filter for a wavelength equal to or more than a light source wavelength.

The image for cell region extraction is an image from which each cell region can be extracted by image processing. Examples thereof include a bright-field photography image acquired through magnification, imaging, and photographing in a bright field in the image acquisition device 1A, and a fluorescence photography image acquired by irradiating a non-stained sample with excitation light of a predetermined wavelength to cause a cell-endogenous fluorescent substance to emit light, and magnifying, imaging, and photographing the emitted light which is fluorescence.

A flow chart for performing image analysis with use of the acquired images is illustrated in FIG. 7. The following processing steps in FIG. 7 are all executed in the image processing device 2A.

First, in Step P1, an image for cell region extraction sent from the image acquisition device 1A is input by the communication I/F 24. Subsequently, in Step P2, a cell region is extracted from the image for cell region extraction, and labeling processing is executed to label each cell.

Meanwhile, in Step P3, an autofluorescence image from the image acquisition device 1A is input by the communication I/F 24. Subsequently, in Step P4, separation of the autofluorescence image is executed for each of an R component, a G component, and a B component. The following processing steps are executed for each of the R, G, and B components after the separation.

In Step P5, a calculation of adding each pixel of the image for cell region extraction and each pixel of the autofluorescence image is performed and, from the resultant addition image, information on a fluorescence color in each cell region and a feature amount related to the cell region are calculated in the subsequent step of Step P6. In Step P7, the feature amount calculated in the processing step described above is output for each cell. The output result can be displayed on the display unit 23 of the image processing device 2A.

This embodiment further includes executing, in Step P8, for the first sample and the second sample as well, acquisition of the feature amount for each cell, calculation of the first autofluorescence information and the second autofluorescence information, and output of a result of comparing the first autofluorescence information and the second autofluorescence information. The comparison result is displayed on the display unit 23.

### (Fluorescence Image Acquisition Method)

As a method of acquiring a fluorescence image, a light source having a center wavelength that is a wavelength of a unicolor from ultraviolet light to visible light as a fluorescence excitation light source wavelength is caused to irradiate a sample in parallel to an optical axis of a lens, or at an angle that is not parallel to but still is not perpendicular to the optical axis, to thereby excite fluorescence of the sample. The fluorescence generated by the excitation is detected through a cut filter installed in front of or behind the lens or the like. As cut filters for excitation light and for fluorescence observation, cut filters for the two wavelengths are selected so that a part of the excitation light source wavelength does not pass through a fluorescence cut filter on an observation side.

### (Bright-Field Image Acquisition Method)

As an example of a method for a case of acquiring a bright-field image in order to identify a position of a cell, the cell size, the cell shape, or the like, a light source having a visible light source wavelength or a white light source having a mixture of visible light wavelengths is caused to irradiate a subject sample in parallel to an optical axis of a lens, or at an angle that is not parallel to but still is not perpendicular to the optical axis, and reflected light from the subject sample, or diffracted light caused by birefringence, and interference therebetween are detected.

(Information Acquired from Image)

The brightness information acquired from the acquired cell image is color information acquired by describing, in a certain color space, information of fluorescence acquired through the method described above, and is coordinates in the color space. The brightness information can be described with use of information on one component out of a red component (an R component), a green component (a G component), and a blue component (a B component) in an RGB color space, which is an example of the color space, or with use of a Lab color space or an HSV color space. For example, the G component serving as information of green fluorescence and the B component serving as information of blue fluorescence are usable as the brightness information of the autofluorescence of FAD⁺ and NAD(P)H described above. A color space other than those may also be used to describe the brightness information.

In addition, the cell shape information obtained from the acquired cell image includes information such as the cell size, the cell shape, and the thickness of the cell. Other information that can be acquired from the image may be used.

As a further embodiment of the present disclosure, there is provided an information processing method for evaluating an immune response of a cell group to a test substance, the information processing method including the steps of: acquiring first autofluorescence information which is autofluorescence information of a first sample prepared by adding the test substance to the cell group; acquiring second autofluorescence information, which is autofluorescence information of a second sample serving as a control for the first sample; and evaluating the immune response of the cell group to the test substance by comparing the first autofluorescence information and the second autofluorescence information.

An information processing device for executing the information processing method and an information processing program for causing an information processing device to execute the information processing method are also provided.

### EXAMPLES

In the following Examples, specific examples of evaluating a cell that responds to stimulation with an antigen by using a PBMC are given. However, reagents and reaction conditions described in the following Examples can be changed and those changes are to be encompassed in the scope of the present disclosure. Accordingly, the following Examples are given for the purpose of helping understanding of the present disclosure, and are not in any way to limit the scope of the present disclosure.

The test substance used in the following two Examples is a peptide that binds directly to an MHC.

### Example 1

### (Thawing and Preculturing of Cells)

Cells that were used were peripheral blood mononuclear cells (PBMCs) (manufactured by Cellular Technology Limited). The PBMCs stored frozen in a liquid nitrogen tank were thawed by following a manual provided by the manufacturer. The thawed cells were suspended in 3 mL of CTL-Test Medium (manufactured by Cellular Technology Limited), and an entire amount was seeded in one well of a six-well plate and cultured overnight in a 37° C., 5% CO₂ incubator.

The CTL-Test Medium used here was added with 200 mM of L-glutamine solution, which was a 1/100 amount of a culture medium capacity of the CTL-Test Medium.

### (Antigen Stimulation Method)

After the cells cultured overnight were collected, 7 mL of PBS was added and 8 minutes of centrifugation was performed at 300 G.

After a supernatant was removed and the cells were suspended in the CTL-Test Medium at a cell concentration of 2.5×10⁶ cells/mL, 100 μL each was seeded in a 96-well U-bottom plate. As test substances, peptide solutions manufactured by Cellular Technology Limited, specifically, Flu Matrix 1 (58-66) Peptide, Influenza A PA (46-54) Peptide, CMV pp65 (495-503) Peptide (all having a peptide concentration of 10 nM) were used, and 100 μL of each of the peptide solutions was added to the wells containing the cells.

The PBMCs used in this experiment are cells that react strongly to Flu Matrix 1 (58-66) Peptide, hardly react to Influenza A PA (46-54) Peptide, and react weakly to CMV pp65 (495-503) Peptide. This information was confirmed in advance by the manufacturer through an ELISpot test.

As a control cell group to which no test substances were added, cells to which 100 μL each of CTL-Test Medium was added instead of a peptide solution were used. After antigen stimulation, the cells were left to stand for 1 hour in a 5% CO₂ incubator. From each well, 200 μL each of cell suspension solution was collected and subjected to 5 minutes of centrifugation at 300 G. After the centrifugation, a supernatant was removed and cell pellets were suspended in 700 μL of PBS. Out of the resultant cell suspension solution, 100 μL was seeded onto a cover glass part (having a diameter of 16 mm) of a 35-mm glass bottom dish, and was used for bright field observation and autofluorescence observation.

### (Acquisition of Bright-Field Image)

A bright-field image was picked up by the image pickup and analysis system 100 through irradiation with light from a 525-nm LED (manufactured by Asahi Spectra Co., Ltd.). For the image pickup unit 13, EOS R5 (a product name) manufactured by Canon Inc. was used and the image was acquired at a shutter speed of 1/400 and an ISO sensitivity of 100.

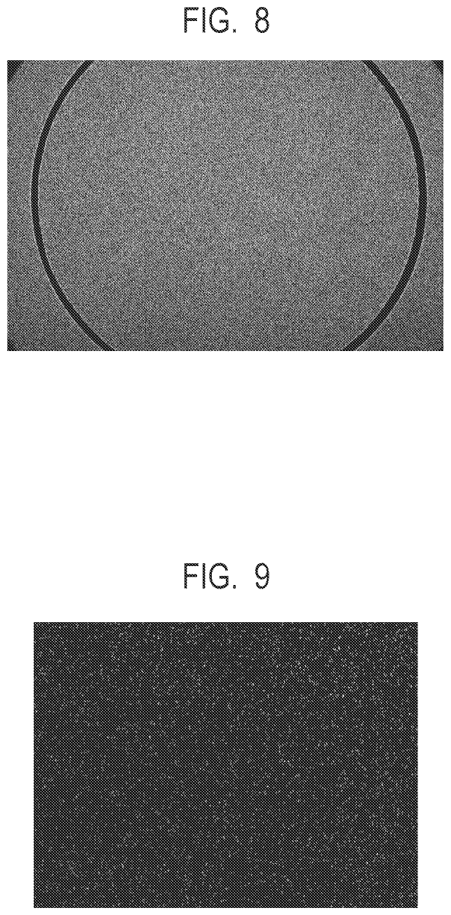

An example of the image photographed by bright-field photography is illustrated in FIG. 8. A viewing field size is approximately 18 mm×12 mm.

### (Acquisition of Autofluorescence Image)

An observation subject was irradiated and excited with light from a light source that is a 365-nm LED (manufactured by Asahi Spectra Co., Ltd.), through a 400-nm short-pass filter, and an image thereof was picked up through a 430-nm long-pass filter (a blue autofluorescence image). The observation subject was irradiated and excited with light from a light source that is a 395-nm LED (manufactured by Asahi Spectra Co., Ltd.), through the 400-nm short-pass filter, and an image thereof was picked up through the 430-nm long-pass filter (a green autofluorescence image). The two images were picked up by the image pickup and analysis system 100. For the image pickup unit 13, EOS R5 was used and the images were acquired at an ISO sensitivity of 12,800.

An example of the photographed blue autofluorescence image is illustrated in FIG. 9. In FIG. 9, an area that is a part of the blue autofluorescence image is cut out, the viewing field size is approximately 6.6 mm×5.25 mm, and 7,642 cells are observed.

### (Method of Extracting and Analyzing Color Information of Autofluorescence Images)

Cell regions were acquired from the bright-field image and labeling was executed. The blue component (B component) and the green component (G component) were acquired from the blue autofluorescence image and the green autofluorescence image, respectively, and then the cell regions acquired from the bright-field image were overlaid to acquire the brightness value of each individual cell. The acquired B component and G component were treated as autofluorescence derived from NADH and autofluorescence derived from FAD⁺, respectively, and a value obtained by dividing a value of the G component of the green autofluorescence image by a value of the B component of the blue autofluorescence image was calculated for each individual cell as the FAD/NADH ratio.

### (Example of Evaluating Reactivity of T Cells in PBMCs to Peptide Antigens)

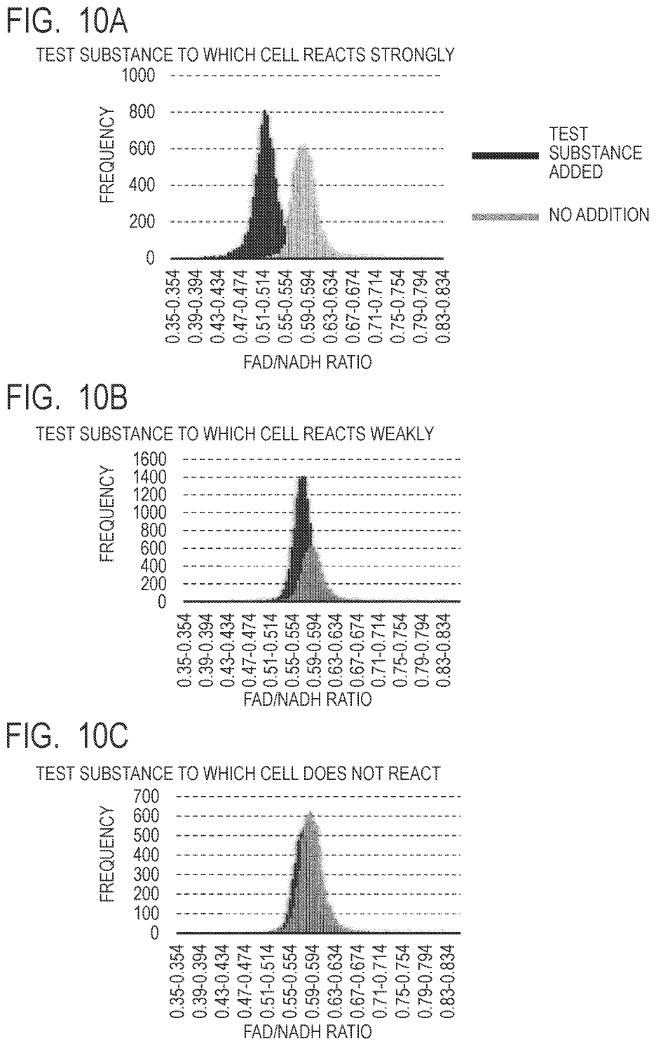

Results of measuring the PBMCs stimulated for 1 hour with the three types of test substances different from one another in terms of reactivity of the PBMCs are shown in FIG. 10A to FIG. 10C. FIG. 10A to FIG. 10C are graphs in which a vertical axis indicates frequency (a cell count) and a horizontal axis indicates the FAD/NADH ratio. In FIG. 10A to FIG. 10C, the black line indicates addition of the test substance and the gray line indicates no addition. FIG. 10A is the result of using a cell group added with the test substance to which the cells react strongly, FIG. 10B is the result of using a cell group added with the test substance to which the cells react weakly, and FIG. 10C is the result of a cell group added with the test substance to which the cells do not react.

A comparison between the cell group added with the test substance (Flu Matrix 1 (58-66) Peptide) used in this Example as a test substance to which the PBMCs reacted strongly and the cell group without the addition revealed that the former was lower in FAD/NADH ratio. The degree of this difference depended on the intensity of reactivity of the PBMCs to the test substance. That is, it has been found out that the intensity of reactivity of an immune cell to a test substance can be known by examining a difference in FAD/NADH ratio between a cell group added with no antigen and a cell group added with an antigen.

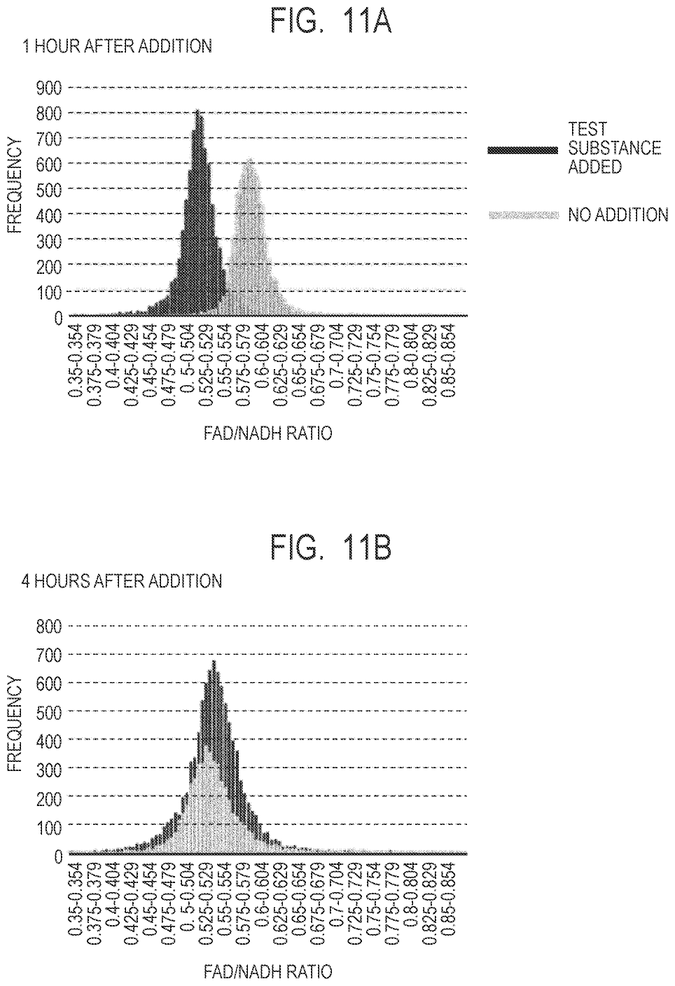

FIG. 11A and FIG. 11B show a result of comparison between the PBMCs 1 hour after stimulation (FIG. 11A) with the test substance to which the PBMCs react strongly and the PBMCs 4 hours after the stimulation (FIG. 11B). In FIG. 11A and FIG. 11B, the black line indicates addition of the test substance and the gray line indicates no addition. A drop in FAD/NADH ratio due to the stimulation with the test substance was observed 1 hour after the stimulation, but the difference diminished substantially to zero by 4 hours after the stimulation. This tells that, in a case of finding out responsiveness of a cell to this test substance, reaction of the cell at a time that is at least less than 4 hours since the stimulation is required to be examined, and that examination of the reaction at an elapse of 1 hour or so is preferred.

### Example 2

### (Thawing of Cells)

PBMCs having reactivity to a test substance that differs from the reactivity in Example 1 were used here. The PBMCs used here were manufactured by Cellular Technology Limited, and thawed by following a manual provided by the manufacturer. Unlike Example 1, preculturing was skipped and the PBMCs were immediately presented to a test for examining reactivity to a test substance.

### (Antigen Stimulation Method)

The thawed PBMCs were suspended in the CTL-Test Medium at a cell concentration of 7.2×10⁶ cells/mL, and 250 μL each was seeded in a 24-well plate. As the test substance, a peptide solution manufactured by Cellular Technology Limited, specifically, CMV pp65 (495-503) Peptide (having a peptide concentration of 10 nM) was used, and 250 μL of this peptide solution was added to each of the wells containing the cells.

The PBMCs used in this experiment are cells that react strongly to CMV pp65 (495-503) Peptide. This information was confirmed in advance by the manufacturer through an ELISpot test.

As a control cell group to which no test substances were added, cells to which 250 μL each of CTL-Test Medium was added instead of a peptide solution were used. After antigen stimulation, the cells were left to stand for 1 hour to 24 hours in a 5% CO₂ incubator. From each well, an entire amount of cell suspension solution was collected and subjected to 5 minutes of centrifugation at 300 G. After the centrifugation, a supernatant was removed and cell pellets were suspended in 1,000 μL of PBS. Further, the resultant cell suspension solution was diluted 5 times with the PBS. Then, 100 μL of the diluted cell suspension solution was seeded onto a cover glass part (having a diameter of 16 mm) of a 35-mm glass bottom dish, and was used for bright field observation and autofluorescence observation.

### (Autofluorescence Image Acquisition and Others)

The subsequent bright-field image acquisition, autofluorescence image acquisition, extraction and analysis of the color information of the autofluorescence images were performed by the same methods as the methods in Example 1.

### (Example of Evaluating Reactivity of T Cells in PBMCs to Peptide Antigen)

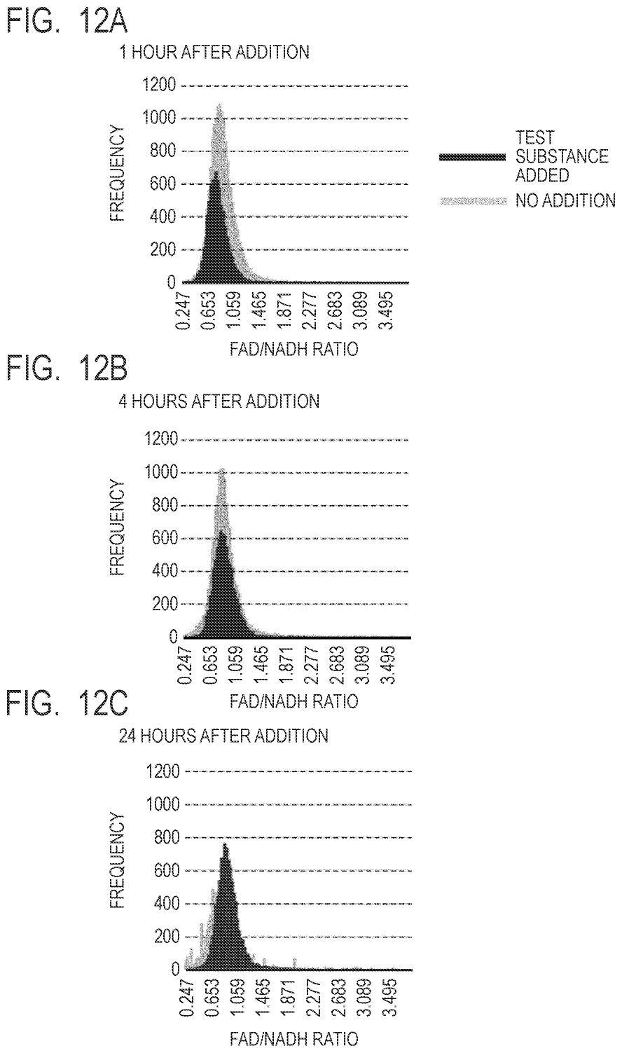

Results of measuring over time the PBMCs stimulated for 1 hour to 24 hours with the test substance to which the PBMCs have strong reactivity are shown in FIG. 12A to FIG. 12C. FIG. 12A to FIG. 12C are also graphs in which a vertical axis indicates frequency (a cell count) and a horizontal axis indicates the FAD/NADH ratio. In FIG. 12A to FIG. 12C, the black line indicates addition of the test substance and the gray line indicates no addition. A result of 1 hour after the stimulation with the test substance, a result of 4 hours after the stimulation with the test substance, and a result of 24 hours after the stimulation with the test substance are shown in FIG. 12A, FIG. 12B, and FIG. 12C, respectively.

The drop in FAD/NADH ratio observed in Example 1 as a decrease compared to the FAD/NADH ratio of the PBMCs with no test substance added thereto was reproduced by 1 hour of stimulation with the test substance (CMV pp65 (495-503) Peptide) used in this experiment as a test substance to which the PBMCs of this experiment reacted strongly. At 4 hours after the stimulation with the test substance, the drop in FAD/NADH ratio was hardly observed as in Example 1. Changes in FAD/NADH ratio were further examined until 24 hours elapsed since the stimulation, but substantially no drop in FAD/NADH ratio was observed in a period from 4 hours after the stimulation with the test substance to 24 hours after the stimulation.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

In evaluating an immune response of a cell group to a test substance, a result can be obtained about 1 hour from the addition of the test substance to the cell group, and testing can accordingly be finished in a shorter time than with the related art.

Another advantage is that reactivity of a cell group to a test substance can be evaluated by simple operation which just includes acquisition of an image with the image pickup and analysis system after the test substance is added and the cell group is left to stand.

Staining operation for rendering intracellular molecules visible in order to measure an endogenous fluorescent substance is unrequired as well, and measurement can be performed without unnecessarily stimulating the cell.

While the present disclosure has been described with reference to embodiments, it is to be understood that the disclosure is not limited to the disclosed embodiments. The scope of the following claims is to be accorded the broadest interpretation so as to encompass all such modifications and equivalent structures and functions.

This application claims the benefit of priority from Japanese Patent Application No. 2023-042238, filed Mar. 16, 2023, which is hereby incorporated by reference herein in its entirety.

What is claimed is:

1. A method of evaluating an immune response of a cell group to a test substance, the method comprising:
preparing a first sample by adding the test substance to the cell group;
preparing a second sample serving as a control for the first sample;
acquiring first autofluorescence information which is autofluorescence information of the first sample;
acquiring second autofluorescence information which is autofluorescence information of the second sample; and
evaluating the immune response of the cell group to the test substance based on a comparison between the first autofluorescence information and the second autofluorescence information.

2. The method according to claim 1, wherein the autofluorescence information comprises information about a brightness value (I_{B}) of blue autofluorescence and a brightness value (I_{G}) of green autofluorescence.

3. The method according to claim 2,
wherein the blue autofluorescence is autofluorescence observed when irradiation is executed with excitation light having a center wavelength in a range of from 320 nm to 370 nm, and
wherein the green autofluorescence is autofluorescence observed when irradiation is executed with excitation light having a center wavelength in a range of from 360 nm to 490 nm.

4. The method according to claim 2, wherein the blue autofluorescence is autofluorescence observed in a range of from 420 nm to 500 nm, and the green autofluorescence is autofluorescence observed in a range of from 500 nm to 580 nm.

5. The method according to claim 1,
wherein the autofluorescence information comprises, for each individual cell of each of the samples, information about a value (I_{G}/I_{B}) of a ratio of a brightness value (I_{B}) of blue autofluorescence and a brightness value (I_{G}) of green autofluorescence, and
wherein the first autofluorescence information and the second autofluorescence information are compared based on distribution of I_{G}/I_{B} of each individual cell of each of the samples.

6. The method according to claim 1, wherein the second sample is prepared by:
omitting addition of the test substance to the cell group,
adding the test substance to the cell group in an amount different from an amount of the test substance in the first sample,
adding a substitute substance for the test substance to the cell group, or
adding the test substance to the cell group, with a time elapsed since the addition varied from an elapsed time of the first sample.

7. The method according to claim 1, further comprising:
acquiring a blue autofluorescence image of each of the samples by irradiating each of the samples with excitation light that has a center wavelength in a range of from 320 nm to 370 nm;
acquiring a green autofluorescence image of each of the samples by irradiating each of the samples with excitation light that has a center wavelength in a range of from 360 nm to 490 nm; and
acquiring autofluorescence information for each cell by analyzing the acquired blue autofluorescence image and green autofluorescence image.

8. The method according to claim 1, wherein the cell group comprises at least one of a T cell, a B cell, or other lymphocytes.

9. The method according to claim 1, wherein the cell group is a group of peripheral blood mononuclear cells (PBMCs).

10. The method according to claim 1, wherein the test substance comprises at least one selected from an antigen, an antigenic protein, an antigenic peptide, a vaccine, and a pathogen.

11. An information processing method for evaluating an immune response of a cell group to a test substance, the information processing method comprising:
an acquiring step of acquiring first autofluorescence information which is autofluorescence information of a first sample, the first sample being prepared by adding the test substance to the cell group;
an acquiring step of acquiring second autofluorescence information which is autofluorescence information of a second sample, the second sample serving as a control for the first sample; and
an evaluating step of evaluating the immune response of the cell group to the test substance by comparing the first autofluorescence information and the second autofluorescence information.

12. An information processing device configured to execute the information processing method of claim 11.

13. A non-transitory storage medium storing an information processing program for causing an information processing device to execute the information processing method of claim 11.

14. An image pickup and analysis system for evaluating an immune response of a cell group to a test substance, the image pickup and analysis system comprising:
an image acquisition device comprising an irradiation unit configured to emit excitation light of 320 nm to 490 nm; and
an image processing device comprising a display unit,
wherein the image acquisition device comprises means for acquiring:
an autofluorescence image of a first sample prepared by adding the test substance to the cell group; and
an autofluorescence image of a second sample serving as a control for the first sample,
wherein the image processing device comprises means for generating, from the autofluorescence images, first autofluorescence information which is autofluorescence information of the first sample and second autofluorescence information which is autofluorescence information of the second sample, and
wherein the display unit is configured to display a result of comparing the first autofluorescence information and the second autofluorescence information.
